(19) United States
(12) Patent Application Publication
(10) Pub. No.: US 2026/0350788 A1
(43) Pub. Date: Nov. 5, 2026

(54) COMPUTER IMPLEMENTED METHOD OF ANALYSING LOCAL PRESSURE VALUES IN A CARDIOVASCULAR SYSTEM OF A BODY OF A PERSON

(71) Applicant: Medis Associated B.V., Leiden (NL)

(72) Inventors: Johannes Petrus Janssen, Leiderdorp (NL); Nikunj DESAI, Eindhoven (NL); Guido Theodoor REBEL, Weesp (NL); Kamran RAHMANI, Delft (NL); Johan Hendrikus Christiaan REIBER, Rotterdam (NL)

(21) Appl. No.: 19/195,298

(22) Filed: Apr. 30, 2025

(51) Int. Cl. G06T 7/00; A61B 5/00; A61B 5/02; A61B 5/021; G06T 7/62; G16H 15/00; G16H 30/40

(52) U.S. Cl. CPC G06T 7/0012; A61B 5/02007; A61B 5/021; A61B 5/7435; A61B 5/7475; G06T 7/62; G16H 15/00; G16H 30/40; G06T 2207/10016; G06T 2207/30096; G06T 2207/30101

(57) ABSTRACT

A computer implemented method of analysing local pressure values in a cardiovascular system of a body of a person. The method comprises receiving a first video stream comprising first frames comprising first image data on a cardiovascular system, receiving a second video stream comprising second frames comprising second image data on the cardiovascular system, presenting to a user, by means of an electronic display screen, at least some of the first frames obtaining a selection of a selected first frame from the first video stream. The method further comprises presenting to a user at least some of the second frames, obtaining a selection of a selected second frame from the second video stream, identifying a vessel of interest, and determining, based on the vessel of interest, a path line from a proximal point in the vessel of interest to a distal point in the vessel of interest.

### FIELD OF THE INVENTION

The various aspects and implementation thereof relate to the field of determining flow data in a blood vessel of a body of a mammal, for example a human body, due to variation of geometrical parameters of the blood vessel, based on images acquired in a non-invasive way.

### BACKGROUND

US9891044B2 discloses a method for determining regions in a blood vessel that may be defined as lesions. Deviations of blood pressure due to stenosis caused by plaque pose a health risk. The deviation, often expressed in a fractional flow reserve, may be calculated on a per-location basis using deviations of the local cross-sectional area or local diameter from a reference value representing a healthy vessel. The reference value may be obtained by means of linear or higher order interpolation or linear regression techniques, or fractal law approach.

Together with the flow velocity of a fluid through the vessel, a value of local deviation of a vessel geometry compared to a reference value allows for accurate, fast and efficient calculation of the fractional flow reserve for every location of the vessel for which geometry data is available. The reference value as well as actual data obtained by measurements may be represented or approximated by functions, allowing accurate and efficient calculation of data over a continuous region of the segment under scrutiny.

For determining various parameters of a cardiovascular system, like fractional flow reserve, flow velocity of blood in a coronary vessel may be required. The flow velocity may be determined as described in “TIMI Frame Count” by C. Michael Gibson e.a. as published in Circulation 1996; 93: 879-888, or simply by the length of the vessel divided by the contrast dye-filling time over the length of the vessel, either in 2D or in 3D as described in “Patent No. [NL 2026715], TITLED [METHOD OF DETERMINING VESSEL FLUID FLOW VELOCITY], by TOBÓN GÓMEZ CATALINA e.a.

### SUMMARY

It is preferred to provide a method with reduced input actions in determining pressure values and lesions in a blood vessel of a body of a living being, and in particular of blood in a coronary vessel of a human being, due to variation in geometry in the blood vessel.

A first aspect provides a computer implemented method of analysing local pressure values in a cardiovascular system of a body of a person. The method comprises receiving a first image data set comprising first frames comprising first image data on a cardiovascular system, presenting to a user, by means of an electronic display screen, at least some of the first frames obtaining a selection of a selected first frame from the first image data set, The method further comprises identifying, in the selected first frame, a vessel of interest, and determining, based on the vessel of interest, a path line from a proximal point in the vessel of interest to a distal point in the vessel of interest.

The computer comprises presenting, to a user, by means of an electronic display screen, the path line in conjunction with the selected first frame, obtaining, from the selected first frame, at least one of model base data as a selection of selected physiological information and a selection of selected morphological information of the vessel of interest, and setting up, based on the model base data and the path line in the vessel of interest, a geometrical model of the vessel of interest.

The computer further comprises determining pressure index values along the path line, based on the geometrical model and physiological values of the vessel of interest, determining, based on the pressure values, a lesion area in the vessel of interest, the lesion area comprising a lesion, presenting to the user, by means of an electronic display screen, lesion image data about the lesion area of at least one lesion in the vessel of interest, and presenting to the user, by means of an electronic display screen, a report comprising data on the lesion areas of the vessel of interest and pressure data.

In one implementation, the method further comprises receiving a second image data set comprising second frames comprising second image data on the cardiovascular system, in response to the obtaining the selection of the selected first frame, presenting to a user, by means of the electronic display screen, at least some of the second frames, obtaining a selection of a selected second frame from the second image data set, identifying the vessel of interest in at least one of the selected first frame and the selected second frame, presenting, to a user, by means of the electronic display screen, the path line in conjunction with at least one of the selected first frame and the selected second frame in the vessel of interest; and obtaining, from at least one of the selected first frame and the selected second frame, at least one of model base data as a selection of selected physiological information and a selection of selected morphological information of the vessel of interest.

In an embodiment, identifying of the vessel of interest comprises identifying vessel boundaries. Using for example edge detection, boundaries of vessels may be detected efficiently.

In another embodiment, the method further comprising receiving, upon presenting the vessel of interest to the user by means of an electronic display screen, via electronic input means from the user, selected vessel of interest information, and selecting a selected vessel of interest in accordance with the selected vessel of interest information. This allows user input in order to enhance precision and reliability of the method.

In yet another embodiment, determining the path line is based on the vessel boundaries of the vessel of interest, based on the model base data. This allows for efficiently determining the location of the path line, in particular if the path line is defined in a centre of the vessel.

In an embodiment, the method further comprising, receiving, upon presenting the path line in the vessel of interest to the user by means of an electronic display screen, via electronic input means, from the user, adjusted path line in the vessel of interest information, and adjusting the path line in the vessel of interest in accordance with the adjusted path line in the vessel of interest information. This allows user input in order to enhance precision and reliability of the method.

In another embodiment, the model base data comprises a selection of physiological information comprising a proximal pressure value in the vessel of interest at the proximal point in the vessel of interest. Such value may serve as a boundary condition for further calculations, providing more efficient calculation of parameters.

In yet another embodiment, the model base data comprises area values of cross-sectional areas of the vessel of interest at locations along the path line. If not the greatest details required for the cross-sectional area to determine, for example, stenotic areas or determined blood flow pressure, a mere value of the area may suffice, instead of an exact geometrical shape.

In an embodiment, the model base data comprises diameter values of cross-sectional areas of the vessel of interest at locations along the path line. This may allow for calculations with improved accuracy as compared to having area values only. In one variation, elliptical cross-sections are used, with a first value for the smallest diameter and a second value for the larger diameter.

In another embodiment, the method further comprises obtaining a diameter threshold or a diameter reduction threshold, and defining a lesion if a cross-sectional area in the geometrical model is below the corresponding diameter threshold or the diameter reduction threshold. Additionally or alternatively, a threshold value for an area of a lumen may be obtained.

In yet another embodiment, the method comprises obtaining a stenosis threshold indicating a particular severity of stenosis. This is an option for detection of stenosis in a model.

In yet another embodiment, the method further comprises receiving, upon presenting lesion image data about the lesion area of at least one lesion in the vessel of interest to the user by means of an electronic display screen, via electronic input means from the user, adjusted lesion image data about the lesion area of at least one lesion in the vessel of interest information, and adjusting lesion image data about the lesion area of at least one lesion in the vessel of interest in accordance with the adjusted lesion image data about the lesion area of at least one lesion in the vessel of interest information. This allows user input in order to enhance precision and reliability of the method.

In an embodiment, the method further comprises receiving, upon presenting at least some of the first frames to the user by means of an electronic display screen, via electronic input means from the user, a selected selection of the selected first frame from the first image data set information; and selecting the selection of the selected first frame from the first image data set in accordance with the selected selection of the selected first frame from the first image data set information. This allows user input in order to enhance precision and reliability of the method.

In another embodiment, the method further comprises receiving, upon presenting at least some of the second frames to the user by means of an electronic display screen, via electronic input means from the user, a selected selection of the selected second frame from the second image data set information, and selecting the selection of the selected second frame from the second image data set in accordance with the selected selection of the selected second frame from the second image data set information. This allows user input in order to enhance precision and reliability of the method.

In yet another embodiment, the selection of the selected second frame from the second image data set is based on the selection of the selected first frame from the first image data set. The selected second frame may for example provide a view of a vessel interest that closely resembles a view of a vessel in the selected first frame. This may allow for more accuracy in building a model and/or executing calculations.

In yet another embodiment, the fourteen frames from each set from the first image data set and the second image data set, comprise of at least two Left Anterior Descending Artery first frames, at least two Left Circumflex Artery first frames, and at least two Right Coronary Artery first frames. In other implementations, three, four, five, six, seven, eight of more frame may be provided for views of one or more of the arteries. In a two-dimensional single view solution, a single frame is used for each of the vessel types. Such single frame may be selected from one, two, three, four, five, six, seven, eight or more than that, for example fourteen frames that are shown on screen or may be selectable.

In yet another embodiment, the second image data comprised by the second image data set is at least twenty-five degrees apart relative to the data comprised by the first image data set, relative to the body. This allows for an enriched imaging process, resulting in a more complete representation of the imaged area. This allows for more accurately building a three-dimensional model.

A second aspect provides a computer programme product comprising computer executable instructions causing an electronic processing unit, when coupled to an electronic memory in which the computer executable instructions are stored, to execute the method according to the first aspect.

A third aspect provides a non-transitory computer readable medium having stored thereon or stored therein the computer programme product according to the second aspect.

A fourth aspect provides an electronic computing device comprising an electronic data processing module arranged to execute the method according to the first aspect.

### BRIEF DESCRIPTION OF THE FIGURES

The various aspects and examples thereof will now be discussed in conjunction with figures. In the figures:

FIG. 1: shows a system for image acquisition and processing;

FIG. 2 A: shows a first part of a flow chart;

FIG. 2 B: shows a second part of a flow chart; and

FIG. 3: shows two images of a cardiovascular structure.

### DETAILED DESCRIPTION

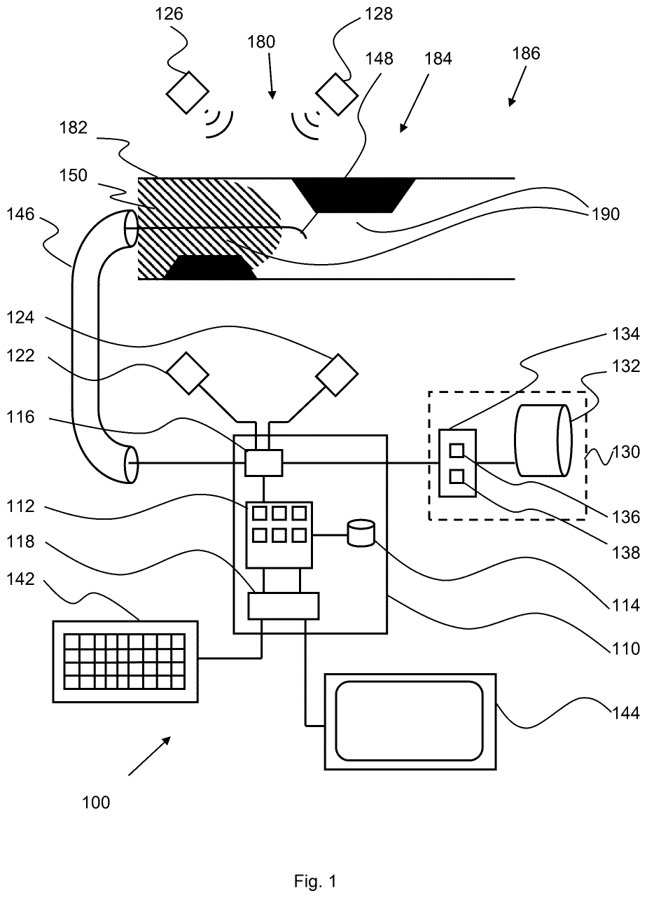

FIG. 1 shows an electronic medical data acquisition and procession system 100 as an example of the second aspect. The system 100 or parts thereof may be found in a cardiac catheterisation laboratory of a clinic or a hospital. The system 100 comprises an X-ray image acquisition module comprising a first X-ray source 126 and a second X-ray source 128, a first X-ray detector 122 arranged to receive X-ray data from the first X-ray source 126 and a second X-ray sensor 124 arranged to receive X-ray data from the second X-ray source 128.

The first X-ray source 126, the second X-ray sensor 124, the first X-ray detector 122 and the second X-ray sensor 124 are arranged to obtain images of a cardiovascular structure 180 under an angle relative to one another, as a first set of image data and a second set of image data. The obtained images are preferably provided in consecutive order, per moment in time when the images were obtained. As such, the first set of image data and the second set of image data may be provided as video streams.

A video stream is a set of images, which images are ordered consecutively in time. the video stream may be provided in a container file, either in compressed or uncompressed form, either in proprietary format or in a standardised format, for example as defined by the MPEG (Motion Pictures Expert Group). In another example, the set of image data may be provided as separate images. The angle is preferably between 25° and 45°, more preferably between 30° and 40°.

Imaging techniques, other than X-ray imaging, may also be employed. The first video stream and the second video stream each comprise at least fourteen frames. The fourteen frames from each set from the first video stream and the second video stream, comprises of at least three Left Anterior Descending Artery frames, at least three Left Circumflex Artery first frames, and at least eight Right Coronary Artery frames.

The first X-ray detector 122 and the second X-ray detector 124 are connected to data acquisition module 116 of an electronic computing device 110. The electronic computing device further comprises a processing unit 112, a storage module 114 and a peripherals I/O controller 118. The processing unit 112, which may be implemented as a microprocessor, microcontroller or other electronic data processing device, is arranged to control the various parts of the electronic computing device 110 and the system 100 and arranged to execute the method according to the first aspect and implementations thereof.

The storage module 114 is arranged for storing data thereon, for example acquired by the computing device 110 from the various other parts of the system 100, either directly or after processing by the processing unit 112. The storage unit 114, as at least partially implemented as a non-transitional storage medium, is further arranged for storing computer executable code which allow the processing unit 112 to execute the method according to the first aspect and implementations thereof.

The system 100 further comprises, in this implementation as an option, a first blood pressure measurement module 130 comprising a pressure cuff 132 and a control unit 134 comprising a pulse sensor 136 and a pressure control unit 138 arranged to control air pressure in the pressure cuff 132 by inflating and deflating the pressure cuff 132.

The system 100 comprises a pressure tip 148 as another blood pressure measurement module, which corresponds to the intracoronary distal pressure. The pressure tip 148, which is connected to a coronary wire, transmits the pressure to the data acquisition module 116, from a coronary artery 182 of the cardiovascular structure 180 as an example of a coronary vessel or blood vessel in general via a catheter 146 inserted in a body of a mammal, such as a human being. Additionally, the tip of the coronary catheter 146, placed into the ostium of the coronary artery under scrutiny 182, sense the proximal pressure into the vessel, which corresponds to the aortic pressure. Furthermore, the catheter 146 may be used to insert contrast dye 150 in the coronary artery 182 or another vessel of a body.

The peripherals I/O controller 118 is arranged to connect the computing device 110 and the various components thereof to input device like a keyboard 142 or a touch screen for receiving data like user input. The peripherals I/O controller 118 is arranged to connect the computing device 110 and the various components thereof to output devices like an electronic display screen 144 and other output devices arranged to provide a user with data on processed or unprocessed data received by the computing device 110.

As shown in FIG. 1, the catheter 146 and the pressure wire 148 are inserted in the coronary artery 182. In the coronary artery 182, narrowings 190 are present. The stenotic areas result in narrowing in the various vessels of the coronary vascular structure 180, which, in turn results in pressure drops at the various stenotic areas.

Subsequently, the pressure drops result in reduced perfusion of myocardial tissues, which leads to reduced physical condition of the person under scrutiny. The cardiovascular structure 180 shown by FIG. 1 may be a hypothetical structure and is not necessarily a representation of an actual anatomical structure.

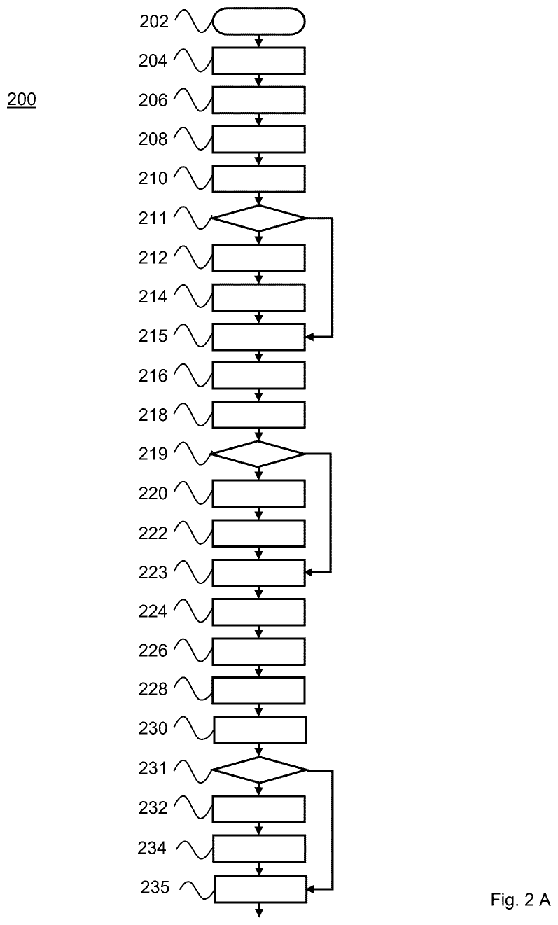

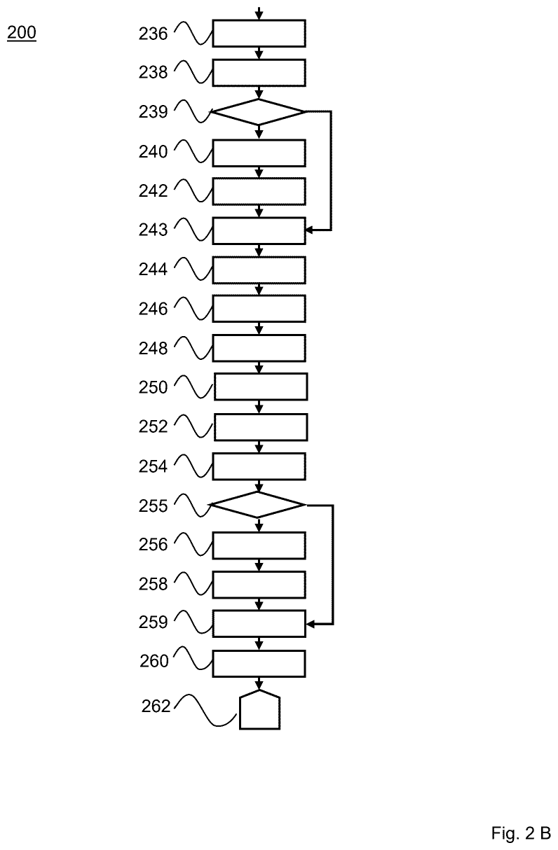

The further functionality of the system 100 and parts thereof discussed above will be further elucidated in conjunction with a flowchart 200 depicted by FIG. 2 A and FIG. 2 B. The procedure depicted by the flow chart 200 is executed by the system 100 and the electronic computing device 110 in particular, controlled by the processing unit 112. The various parts of the flowchart 200 are briefly summarised below.
202 Start procedure
204 Receive first video stream
206 Receive second video stream
208 Identify a first default selected frame
210 Present to user at least some of the first frames
211 User input received?
212 Receive user input
214 Select the selection of the first frame based on user input
215 Select the selection of the first frame
216 Identify a second default selected frame
218 Present to user at least some of the second frames
219 User input received?
220 Receive user input
222 Select the selection of the second frame based on user input
223 Select the selection of the second frame
224 Identify at least one of the three major coronary vessel types
226 Identify the vessel boundaries of vessels
228 Identify a vessel of interest
230 Present to user the vessel of interest
231 User input received?
232 Receive user input
234 Select the selected vessel of interest based on user input
235 Select the selected vessel of interest
236 Determine a path line in the vessel of interest
238 Present to user the path line in the vessel of interest
239 User input received?
240 Receive user input
242 Adjust adjusted path line in the vessel of interest based on user input
243 Select the path line in the vessel of interest
244 Obtain physiological information morphological information of the vessel of interest
246 Set up a geometrical model of the vessel
248 Obtain a diameter threshold or a diameter reduction threshold, area threshold or a stenosis threshold
250 Define a lesion area
252 Determine a lesion area in the vessel of interest
254 Present to user lesion image data
255 User input received?
256 Receive user input
258 Adjust adjusted lesion image data based on user input
259 Select the lesion area in the vessel of interest
260 Present to user patient report
262 End procedure

The procedure starts in step 202. The procedure continues in step 204 for receiving a first video stream, wherein the first video stream comprises first frames comprising first image data of the cardiovascular structure 180 shown by FIG. 1. The first video streams are acquired over time by means of the first X-ray detector 122, while the contrast dye 150 progresses through the coronary artery 182 over time.

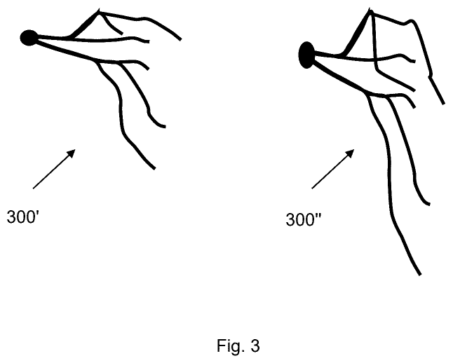

FIG. 3 shows, at the left, a first image 300′ as an example of the last image or one of the last images of the first video stream, in which the vessels of the cardiovascular structure 180 are filled with the contrast dye 150. The first video stream may also be referred to as the first series of images.

In step 206, a second video stream is received by the electronic computing device 110 depicted in FIG. 1. The second video stream comprises second frames comprising second image data on the cardiovascular structure 180. The second image data, comprised by the second video stream, is at least twenty-five degrees apart relative to the data comprised by the first video stream, relative to the body.

The second video stream is preferably acquired by means of the first X-ray detector 124, in analogous fashion as the first video stream is obtained. The first video stream is preferably obtained at the same moment as the second video stream is obtained. FIG. 3 shows, at the left, a second image 300″ as an example, a last image or one of the last images of the second video stream. This second video stream may also be referred to as the second series of images, in which the vessels of the cardiovascular structure 180 are filled with the contrast dye 150.

In this example, wherein two X-ray sources and two X-ray image sensors are available in a laboratory, step 204 and 206 may take simultaneously. In case only one X-ray source and only one X-ray image sensor are available in a laboratory, the second series of images maybe acquired after the first series of images has been acquired, with a second dose of contrast dye 150 to be inserted in the cardiovascular structure 180. Thus, step 206 will take place after step 204.

In step 208, a first default selected frame from the first frames is identified for constructing a geometrical model of the cardiovascular structure 180 or in any case of the coronary artery 182 under scrutiny. Preferably, the selected frame comprises image data of the heart in end-diastole position.

The first frame may be selected as disclosed by European patent application published with number EP3660858. In step 210 at least some of the first frames, including the first default selected frame, are presented to the user by means of an electronic display screen 144 as shown in FIG. 1.

A default selected frame is an optimal frame computed by the system and the electronic computing device. The user may select from the selected first frames from the first video stream, including the first default selected frame, from the first video stream. The user may deviate from the default selected frame, by for example selecting a different frame. In this case, the user may select from the selected first frames from the first video stream, excluding the first default selected frame. The selection may be provided to the computing device 110 by means of the keyboard 142 or other user input means as user input.

In step 211, the procedure checks whether any user input is received. If this the case, the procedure continues to step 212 in which the user input is received via electronic means. If not, the procedure branches forward to step 215. The user input comprises a selected selection of the selected first frame from the first video stream information. In step 214, the selection of the selected first frame from the first video stream in accordance with the selected selection of the selected first frame from the first video stream information is selected.

In step 215, the selection of the selected first frame from the first video stream is selected. Such selection, as in step 215, may be part of an automated process or may be retrieved by means of the keyboard 142 or a touch screen, another input device, or a combination of two or more thereof.

The geometrical model may comprise only a centreline of the coronary artery 182, a volumetric model, another model or a combination or two or more thereof. The geometrical model is constructed such that a length of the coronary artery 182 may be determined, preferably a true or natural length. In on example, the volumetric model is provided by providing elliptical approximations of vessel cross-sections along the centreline.

The area of the ellipses preferably approximates the actual area of a cross-section of the vessel at the corresponding location in the actual vessel. A length of the coronary artery 182 which may be obtained from a single, two-dimensional image of one of the two series, may not be the actual length of coronary artery 182 as such arteries usually do not follow a straight path parallel to the image plane.

In step 216, based on the selection of the selected first frame from the first video stream a second default selected frame from the second frames from the second video stream is identified. Thus, the selection of the selected second frame from the second video stream is based on the selection of the selected first frame from the first video stream.

For each selection of a selected first frame from the first video stream, a corresponding selection of a selected second frame from the second video stream will be presented to the user. In another embodiment, a selection of a selected second frame from the second video stream presented to the user may not be corresponding to the selected first frame from the first video stream.

In step 218 at least some of the second frames, including the second default selected frame, are presented to the user by means of an electronic display screen 144. The user then may select from the selected second frames from the second video stream, including the second default selected frame, from the second video stream. However, in case the user deviates from selecting the first default selected frame, by for example selecting a different frame, during the selection of the first frame, then the user may take the same deviation from the second default selected frame when selecting the second frame from the second video streams.

The user then may select from the selected second frames from the second video stream, excluding the first default selected frame. Additionally, the order of the second selected frame by the user may correspond to the same deviation applied to the order of the first selected frame by the user.

In step 219, the procedure checks whether any user input is received. If this is the case, the procedure continues to step 220 in which the user input is received via electronic means. If not, the procedure branches forward to step 223. The user input comprises a selected selection of the selected second frame from the second video stream information.

In step 222, the selection of the selected second frame from the second video stream in accordance with the selected selection of the selected second frame from the second video stream information is selected. In step 223, the selection of the selected second frame from the second video stream is selected. Such selection may be part of an automated process or may be retrieved by means of the keyboard 142 or a touch screen, another input device, or a combination of two or more thereof.

In step 224, at least one of the three major coronary vessel types by means of image recognition model is identified. The three major coronary vessel types comprises Left Anterior Descending Artery (LAD), Left Circumflex Artery (LCX), Right Coronary Artery (RCA). The first image data and the second image data each comprise at least three major coronary vessel types.

In step 226, the boundaries of a vessels are identified by means of a computational method configured to detect the edges of the vessel of the vessel of interest. The computational method is configured to determine a start point and an end point of the vessel of interest. Such a computational method may comprise use of an edge detection method algorithm. Alternatively or additionally, it may comprise a pattern recognition algorithm.

In step 228, a vessel of interest is then identified in at least one of the selected first frame and the selected second frame. Identifying the vessel of interest may comprise identifying vessel boundaries

In step 230, the vessel of interest is presented to the user by means of an electronic display screen 144. In step 231, the procedure checks whether any user input is received. If this is the case, the procedure continues to step 232 in which the user input is received via electronic means. If not, the procedure branches forward to step 235.

In step 232, the user input, comprising selected vessel of interest and/or a start point and an end point of the vessel of interest information, is received. In step 234, a selected vessel of interest, and/or a selected start point and end point of the vessel of interest, in accordance with the selected vessel of interest, and/or a start point and an end point of the vessel of interest information, is selected. In step 235, a vessel of interest and/or a start point and an end point of the vessel of interest is selected. Such selection may be part of an automated process or may be retrieved by means of the keyboard 142 or a touch screen, another input device, or a combination of two or more thereof.

In step 236, a path line, from a proximal point in the vessel of interest to a distal point in the vessel of interest, is determined based on the identified vessel of interest. Determining the path line is based on the vessel boundaries, of the vessel of interest, based on the model base data. The path line may be the same as the centreline.

In step 238, the path line, and a start point and an end point of the path line, in the vessel of interest in conjunction with at least one of the selected first frame and the selected second frame in the vessel of interest is presented to the user by means of an electronic display screen 144.

In step 239, the procedure checks whether any user input is received, as described above. If this is the case, the procedure continues to step 240 in which the user input is received via electronic means. If not, the procedure branches forward to step 243.

In step 240, the user input, comprising adjusted path line and/or a start point and end point of the path line in the vessel of interest information, is received. In step 242, the path line in the vessel of interest and/or the start point and end point of the path line in the vessel of interest is adjusted in accordance with the adjusted path line and/or a start point and end point of the path line in the vessel of interest information, received as user input.

In step 243, a path line in the vessel of interest and/or a start point and an end point of the path line in the vessel of interest is selected. Such selection may be part of an automated process or may be retrieved by means of the keyboard 142 or a touch screen, another input device, or a combination of two or more thereof.

In step 244 at least one of model base data as a selection of selected physiological information and a selection of selected morphological information of the vessel of interest is obtained, from at least one of the selected first frame and the selected second frame.

The model base data comprises a selection of physiological information comprising a proximal pressure value in the vessel of interest at the proximal point in the vessel of interest. The model base data further comprises area values of cross-sectional areas of the vessel of interest at locations along the path line. The model base data further comprises diameter values of cross-sectional areas of the vessel of interest at locations along the path line. The pressure value may be an absolute pressure value, a relative pressure value or a pressure index value.

In step 246, based on the model base data and the path line in the vessel of interest a geometrical model of the vessel of interest is set up. The geometrical model classifies a vessel of interest, as such the model provides an approximation of a physical shape of a vessel. Based on the geometrical model and physiological values of the vessel of interest, pressure values along the path line can be determined.

The geometrical model comprises a selection of selected physiological information and a selection of selected morphological information of at least one lesion. The physiological information comprises hemodynamic parameters, such as a proximal pressure index value in at least one lesion from a proximal point to a distal point of the lesion and blood flow rate in a vessel.

The morphological information comprises information with respect to dimensions of a lesion, for example the diameter and length of a lesion, and the cross-section shape of a lesion. The geometrical model comprises definitions of cross-sectional areas of at least one lesion at locations along the path line.

In step 248 a diameter threshold value, a diameter reduction threshold value, area threshold value, or a stenosis threshold value, is obtained. Alternatively or additionally, threshold values are obtained for lumen area. In step 250, a lesion area is defined if a cross-sectional area in the geometrical model is below the diameter threshold. In this 250 step, a start point and an end point of at least one lesion in vessel of interest is also defined. Another method of determining a lesion area in the vessel of interest is based on the pressure values.

A lesion area may be defined as an area if there is a pressure drop within a particular length of the vessel of interest larger than a threshold length value. Additionally or alternatively, a lesion area—in terms of length of a vessel, may be defined as an area over which at least one of diameter, area, cross-section is less than a reference value to the entity with more than a threshold value obtained as discussed above.

The reference value may be obtained by interpolation of a proximal value of the actual entity under scrutiny between the value at a proximal point of the vessel of interest and a distal value at a distal point of the vessel of interest. The proximal point and distal point of the vessel may be the start point and the end point, but other points may be selected as well.

The interpolation may be linear, quadratic, higher order, other, or a combination thereof. The reference value may, additionally or alternatively, be based on data obtained from other sources, including empirical data from other subjects, either directly or indirectly, for example via a trained machine.

In step 252, a lesion area in the vessel of interest is determined based on the pressure values, wherein the lesion area comprises a lesion. In step 254, the lesion image data about the lesion area of at least one lesion in the vessel of interest is presented to the user by means of an electronic display screen.

In step 255, the procedure checks whether any user input is received. If this is the case, the procedure continues to step 256 in which the user input is received via electronic means. If not, the procedure branches forward to step 259. In step 256, the user input, comprising adjusted lesion image data about the lesion area of at least one lesion in the vessel of interest information and/or a start point and an end point of at least one lesion in the vessel of interest information, is received.

In step 258, the lesion image data about the lesion area of at least one lesion in the vessel of interest and/or a start point and an end point of at least one lesion in the vessel of interest is adjusted in accordance with the adjusted lesion image data about the lesion area of at least one lesion in the vessel of interest information and/or a start point and an end point of at least one lesion in the vessel of interest information.

In step 259, at least one lesion in the vessel of interest and/or a start point an end point of at least one lesion in the vessel of interest is selected. Such selection may be part of an automated process or may be retrieved by means of the keyboard 142 or a touch screen, another input device, or a combination of two or more thereof. Lesion image data comprises data with respect to the length of the lesion, the diameter of the lesion, pressure values along the length of the lesion.

In step 260, a report, comprising data on the lesion areas of the vessel of interest and pressure data, is presented to the user by means of an electronic display screen 144. Subsequently, in step 262, the procedure ends.

Herein, the invention is described with reference to specific examples of embodiments of the invention. It will, however, be evident that various modifications and changes may be made therein, without departing from the essence of the invention.

Other modifications, variations, and alternatives are also possible. The specifications, drawings and examples are, accordingly, to be regarded in an illustrative sense rather than in a restrictive sense.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

In the description above, it will be understood that when an element such as layer, region or substrate is referred to as being “on”, “onto” or “connected to” another element, the element is either directly on or connected to the other element, or intervening elements may also be present.

Furthermore, the invention may also be embodied with less components than provided in the embodiments described here, wherein one component carries out multiple functions. Just as well may the invention be embodied using more elements than depicted in the Figures, wherein functions carried out by one component in the embodiment provided are distributed over multiple components.

It is stipulated that the reference signs in the claims do not limit the scope of the claims, but are merely inserted to enhance the legibility of the claims.

1. A computer implemented method of analysing local pressure values in a cardiovascular system of a body of a person, the method comprising:
receiving a first image data set comprising first frames comprising first image data on a cardiovascular system;
presenting to a user, by means of an electronic display screen, at least some of the first frames;
obtaining a selection of a selected first frame from the first image data set;
identifying, in the selected first frame, a vessel of interest;
determining, based on the vessel of interest, a path line from a proximal point in the vessel of interest to a distal point in the vessel of interest;
presenting, to a user, by means of an electronic display screen, the path line in the vessel of interest in conjunction with the selected first frame;
obtaining, from the selected first frame, at least one of model base data as a selection of selected physiological information and a selection of selected morphological information of the vessel of interest;
setting up, based on the model base data and the path line in the vessel of interest, a geometrical model of the vessel of interest;
determining pressure values along the path line, based on the geometrical model and physiological values of the vessel of interest;
determining, based on the pressure values, a lesion area in the vessel of interest, the lesion area comprising a lesion;
presenting to the user, by means of an electronic display screen, lesion image data about the lesion area of at least one lesion in the vessel of interest;
presenting to the user, by means of an electronic display screen, a report comprising data on the lesion areas of the vessel of interest and pressure data.

2. The method according to claim 1, further comprising:
receiving a second image data set comprising second frames comprising second image data on the cardiovascular system;
in response to the obtaining the selection of the selected first frame, presenting to a user, by means of the electronic display screen, at least some of the second frames;
obtaining a selection of a selected second frame from the second image data set;
identifying the vessel of interest in at least one of the selected first frame and the selected second frame;
presenting, to a user, by means of the electronic display screen, the path line in conjunction with at least one of the selected first frame and the selected second frame in the vessel of interest; and
obtaining, from at least one of the selected first frame and the selected second frame, at least one of model base data as a selection of selected physiological information and a selection of selected morphological information of the vessel of interest.

3. The method according to claim 1, wherein identifying of the vessel of interest comprises identifying vessel boundaries.

4. The method according to claim 3, further comprising:
receiving, upon presenting the vessel of interest to the user by means of an electronic display screen, via electronic input means from the user, selected vessel of interest information; and
selecting a selected vessel of interest in accordance with the selected vessel of interest information.

5. The method according to claim 3, wherein determining the path line is based on the vessel boundaries of the vessel of interest, based on the model base data.

6. The method according to claim 1, further comprising:
receiving, upon presenting the path line in the vessel of interest to the user by means of an electronic display screen, via electronic input means, from the user, adjusted path line in the vessel of interest information; and
adjusting the path line in the vessel of interest in accordance with the adjusted path line in the vessel of interest information.

7. The method according to claim 1, wherein the model base data comprises a selection of physiological information comprising a proximal pressure value in the vessel of interest at the proximal point in the vessel of interest.

8. The method according to claim 1, wherein the model base data comprises area values of cross-sectional areas of the vessel of interest at locations along the path line.

9. The method according to claim 1, wherein the model base data comprises diameter values of cross-sectional areas of the vessel of interest at locations along the path line.

10. The method according to claim 9, further comprising:
obtaining a diameter threshold or a diameter reduction threshold; and
defining a lesion if a cross-sectional area in the geometrical model is below the diameter threshold or the diameter reduction threshold.

11. The method according to claim 1, further comprising:
receiving, upon presenting lesion image data about the lesion area of at least one lesion in the vessel of interest to the user by means of an electronic display screen, via electronic input means from the user, adjusted lesion image data about the lesion area of at least one lesion in the vessel of interest information; and
adjusting lesion image data about the lesion area of at least one lesion in the vessel of interest in accordance with the adjusted lesion image data about the lesion area of at least one lesion in the vessel of interest information.

12. The method according to claim 1, further comprising:
receiving, upon presenting at least some of the first frames to the user by means of an electronic display screen, via electronic input means from the user, a selected selection of the selected first frame from the first image data set information; and
selecting the selection of the selected first frame from the first image data set in accordance with the selected selection of the selected first frame from the first image data set information.

13. The method according to claim 1, further comprising:
receiving, upon presenting at least some of the second frames to the user by means of an electronic display screen, via electronic input means from the user, a selected selection of the selected second frame from the second image data set information; and
selecting the selection of the selected second frame from the image data set stream in accordance with the selected selection of the selected second frame from the second image data set information.

14. The method according to claim 1, wherein the selection of the selected second frame from the second image data set is based on the selection of the selected first frame from the first image data set.

15. The method according to claim 1, wherein the fourteen frames from each set from the first image data set and the second image data set, comprise of at least three Left Anterior Descending Artery first frames, at least three Left Circumflex Artery first frames, and at least eight Right Coronary Artery first frames.

16. The method according to claim 1, further comprising identifying at least one of the three major coronary vessel types by means of image recognition model, the three major coronary vessel types comprising:
Left Anterior Descending Artery (LAD)
Left Circumflex Artery (LCX)
Right Coronary Artery (RCA).

17. The method according to claim 1, wherein the second image data, comprised by the second image data set, is at least twenty-five degrees apart relative to the data comprised by the first image data set, relative to the body.

18. A computer programme product comprising computer executable instructions causing an electronic processing unit, when coupled to an electronic memory in which the computer executable instructions are stored, to execute the method according to claim 1.

19. A non-transitory computer readable medium having stored thereon or stored therein the computer programme product according to claim 18.

20. An electronic computing device comprising an electronic data processing module arranged to execute the method according to claim 1.
